(19) United States
(12) Patent Application Publication
(10) Pub. No.: US 2026/0144740 A1
(43) Pub. Date: May 28, 2026

(54) COSMETIC ACTIVE INGREDIENT DERIVED FROM A NON-VOLATILE FRACTION OF CANARIUM EXUDATE AND USES THEREOF

(71) Applicant: FLORE SCOLA, Saint-Gely-du-Fesc (FR)

(72) Inventors: Michel BONNARD, Montpellier (FR); Jean-Pierre SOLIGNAC, Saint-Mathieu-de-Treviers (FR)

(21) Appl. No.: 19/123,498

(22) PCT Filed: Oct. 26, 2023
(86) PCT No.: PCT/EP2023/079958

§ 371 Date: Jul. 15, 2025

(30) Foreign Application Priority Data
Oct. 26, 2022 (FR) FR2211123

(51) Int. Cl. A61K 8/9789; A61Q 19/00

(52) U.S. Cl. CPC A61K 8/9789; A61Q 19/008

(57) ABSTRACT

The invention relates principally to an active ingredient intended to confer an at least non-therapeutic cosmetic effect, said ingredient comprising an extract consisting of a non-volatile fraction of exudate of Canarium, preferably of Canarium schweinfurthii.

The invention also relates to the uses of an active ingredient derived from a non-volatile fraction of Canarium exudate as a cosmetic agent for use by topical application conferring a repairing and/or anti-inflammatory effect, but also for combating acne, excess sebum and blackheads.

### TECHNICAL FIELD

The invention relates to the cosmetic field by proposing a natural cosmetic ingredient derived from a non-volatile fraction of exudate, or oleoresin, of Canarium.

The invention mainly relates to such a cosmetic ingredient, to its preparation process and uses thereof for use by topical application conferring a healing and/or anti-inflammatory effect, but also for combating acne, the presence or appearance of blackheads or even excess sebum secretion.

### PRIOR ART AND DISADVANTAGES OF THE PRIOR ART

In the cosmetic field and more particularly for cosmetic products applied to the skin, consumers are increasingly seeking products formulated from ingredients of natural origin that are respectful of the environment and the user, in order to reflect their aspirations for a healthy lifestyle as well as their environmental and ethical concerns. Additionally, such products do not contain ingredients originating notably from petrochemicals, limiting feelings of discomfort, comedogenic reactions or even the appearance of redness and are more eco-responsible. Thus, products based on natural ingredients of plant origin, respectful of the environment, constitute a strong expectation on the part of consumers who are always looking for innovative well-being products.

As such, there is a need for new cosmetic products based on natural ingredients, even vegan ingredients, having healing and anti-inflammatory properties, but also making it possible to combat acne, excess sebum and/or the presence of blackheads.

### OBJECTIVE OF THE INVENTION

In this context, the invention proposes a new active ingredient or cosmetic active principle, in particular dermo-cosmetic, of plant origin, obtained in an ethical and eco-responsible manner, the preparation of which is easily reproducible, inexpensive, and which has notable skin repair and anti-inflammatory properties, in addition, it is effective against acne, excess sebum, and blackheads.

### STATEMENT OF THE INVENTION

To meet this need, the inventors were interested in a natural and original raw material to develop such an active ingredient, namely Canarium, in particular a Canarium exudate.

For this purpose, the active ingredient of the invention is substantially characterized in that it comes from a non-volatile fraction of Canarium exudate. Also, according to a first aspect, the invention relates to a cosmetic active ingredient comprising at least one Canarium extract, said extract consisting of the non-volatile fraction of Canarium exudate,

The non-volatile fraction of Canarium exudate is therefore an extract of Canarium exudate and comprises a mixture of molecules. Preferably, the fraction comprises at least one triterpene. More preferably, the fraction consists of a mixture of triterpenes.

According to a preferred object, the fraction comprises at least one neutral triterpene and/or at least one acidic triterpene. Other triterpenes may also be present, such as acid esters.

Preferentially, the neutral triterpene is selected from lanosterol, β-amyrin, α-amyrin, lupeol, uvaol, erythrodiol and combinations thereof. More preferentially, the fraction comprises a mixture of triterpenes, namely lanosterol, β-amyrin, α-amyrin, lupeol, uvaol and erythrodiol.

When the fraction comprises an acidic triterpene, this is preferentially selected from elemolic acid, β-elemonic acid, tsugaric acid A, ursolic acid, oleanolic acid and combinations thereof.

According to a particularly preferred object of the invention, the extract, that is, the non-volatile fraction of Canarium exudate comprises at least lanosterol, β-amyrin and α-amyrin, elemolic acid, β-elemonic acid, tsugaric acid A, lupeol, uvaol, erythrodiol, ursolic acid and oleanolic acid.

In a particularly preferred manner, the ingredient is derived from a non-volatile fraction of Canarium schweinfurthii, which confers enhanced efficacy in skin healing. Also, the ingredient according to the invention preferentially comprises an extract which is a non-volatile fraction of exudate from the species Canarium schweinfurthii, Elemier or African Elemi.

The ingredient of the invention may also comprise the following optional features considered in isolation or according to all possible technical combinations:
the ingredient is derived from a hydrodistillation or vapodistillation residue of Canarium exudate, in particular the fraction is a hydrodistillation or vapodistillation residue of Canarium exudate, and/or
the ingredient has a lanosterol content at least equal to 55 milligrams per gram, and/or
the ingredient has a β-amyrin content at least equal to 85 milligrams per gram, and/or
the ingredient has an α-amyrin content at least equal to 215 milligrams per gram, and/or
the ingredient has an elemolic acid content at least equal to 20 milligrams per gram, and/or
the ingredient has a β-elemonic acid content at least equal to 45 milligrams per gram, and/or
the ingredient has a tsugaric acid A content at least equal to 25 milligrams per gram.
The extract from the hydrodistillation or vapodistillation residue of Canarium schweinfurthii exudate is preferentially an alcoholic or hydroalcoholic extract.

Finally, in a particularly advantageous manner, the active ingredient according to the invention is capable of being obtained by a method comprising the following steps:
hydrodistillation or vapodistillation of Canarium exudate, in particular Canarium schweinfurthii, and
obtaining a hydrodistillation or vapodistillation residue forming the non-volatile fraction of Canarium, and
extracting the non-volatile fraction obtained with the solvent, said solvent preferably being a polar solvent other than water, or a non-polar solvent, and
collecting solid residues by filtration or centrifugation, and
evaporating the filtrate or supernatant to produce an active ingredient powder.

The invention also relates to a cosmetic composition characterized in that it comprises at least one ingredient according to any one of the objects previously described, preferentially the ingredient represents at least 0.1% by weight of the total weight of the composition.

The invention further relates to the use of a non-volatile fraction of Canarium exudate as a cosmetic agent for use by topical application conferring a repairing and/or anti-inflammatory effect.

According to another object, the present invention also relates to the ingredient according to any one of the aforementioned objects, for use thereof for its healing effects on the skin, or for improving skin repair.

The ingredient according to the invention is also intended to be used for its anti-inflammatory effects, notably to combat inflammation of skin cells and/or sebocytes.

According to another object, the invention relates to the ingredient according to the invention for use thereof on the skin to prevent and/or combat acne and/or combat excess sebum secretion.

The use of the invention may also comprise the following optional features considered in isolation or according to all possible technical combinations:
the non-volatile fraction is derived from a residue of hydrodistillation or vapodistillation of Canarium exudate.
the non-volatile fraction of Canarium exudate has a lanosterol content at least equal to 55 milligrams per gram, and/or
the non-volatile fraction of Canarium exudate has a β-amyrin content at least equal to 85 milligrams per gram, and/or
the non-volatile fraction of Canarium exudate has an α-amyrin content at least equal to 215 milligrams per gram, and/or
the non-volatile fraction of Canarium exudate has an elemolic acid content at least equal to 20 milligrams per gram, and/or
the non-volatile fraction of Canarium exudate has a β-elemonic acid content at least equal to 45 milligrams per gram, and/or
the non-volatile fraction of Canarium exudate has a tsugaric acid A content at least equal to 25 milligrams per gram.
the non-volatile fraction of Canarium exudate is derived from a hydrodistillation or vapodistillation residue of Canarium schweinfurthii.

Finally, the use of the ingredient according to the invention is also aimed at combating blackheads, including the appearance of blackheads.

The invention relates finally to a method for manufacturing an ingredient as previously defined which comprises at least the steps of:
hydrodistillation or vapodistillation of Canarium exudate
obtaining a hydrodistillation residue forming a non-volatile fraction of Canarium,
extracting the hydrodistillation residue from the solvent, said solvent being a solvent, preferably polar other than water or non-polar, and
obtaining the ingredient.

The method of the invention may also comprise the following optional features considered in isolation or according to any possible technical combinations:
the solvent extraction operation is carried out at least twice, preferably three times.
the method further comprises at least one step of removing the solvent, for example by vacuum evaporation.
the Canarium exudate is preferentially an exudate from Canarium schweinfurthii.

### DETAILED DESCRIPTION OF THE INVENTION

The invention is based on the discovery of the healing and anti-inflammatory properties of a non-volatile fraction of Canarium exudate, in particular from Canarium schweinfurthii. Additionally, this fraction is also surprisingly effective in combating acne, excess sebum or even the presence or appearance of blackheads. This non-volatile fraction thus makes it possible to manufacture an active ingredient or active principle with such properties, namely healing and anti-inflammatory, combating acne, excess sebum secretion and blackheads. The field of application of the ingredient is the non-therapeutic cosmetic field, more particularly for use by topical application with a healing, skin-repair and anti-inflammatory effect.

For the purposes of the invention, “cosmetic active principle” or “cosmetic active ingredient” means at least one molecule, preferentially a set of molecules, notably triterpenes, having an effect on the skin.

For the purposes of the present invention, “extract” of a raw material X means any molecule or mixture of at least two molecules obtained from a raw material X, regardless of the method of extraction of said molecule or said molecules. Preferentially the raw material is Canarium exudate. This can be an extract obtained by hydrodistillation or vapodistillation of the Canarium exudate, allowing separation of the volatile fraction and the non-volatile fraction of Canarium exudate, for example the non-volatile fraction of a residue from hydrodistillation or vapodistillation of Canarium exudate, more preferentially, hydrodistillation or vapodistillation is followed by solvent extraction.

Also, within the scope of the invention, the non-volatile fraction of Canarium exudate is taken to mean the fraction that does not comprise the volatile compounds that form the essential oil. These two fractions, volatile and non-volatile, can be separated by hydrodistillation or vapodistillation of the exudate. The result is the essential oil on the one hand, and the non-volatile fraction, the object of the present invention, on the other hand. The use of a Canarium extract gives the resulting ingredient an eco-responsible character, notably due to the use of a renewable plant resource and its non-destructive collection. The use of a hydrodistillation or vapodistillation residue also has an eco-responsible character since residues from the production of essential oils by hydrodistillation or vapodistillation of a plant material are normally considered as waste, the invention ensuring, on the contrary, the re-use of this co-product.

The exudate, or oleoresin, used within the scope of the invention comes from plants of the Canarium genus which includes around a hundred tree species, and can be recovered by non-destructive incision of the tree bark. The invention relates more particularly, but is not limited to, the non-volatile fraction of Canarium luzonicum, whose fragrant resin is known as Asian elemi, to the non-volatile fraction of Canarium madagascariense, and to the non-volatile fraction of Canarium schweinfurthii (African elemi). Preferred will be, but not limited to, Canarium schweinfurthii for the enhanced efficacy of its properties as shown in Example 4.

The method for preparing the ingredient of the invention has two main steps: a hydrodistillation or vapodistillation operation, and a solvent extraction operation which can be repeated several times.

The exudate, or oleoresin, of Canarium schweinfurthii, or of Canarium luzonicum or of Canarium madagascariense is removed by non-destructive incision. The exudate is placed at low temperature, for example −20° C., and split into pieces.

For hydrodistillation, the pieces of exudate are introduced into the flask of the hydrodistillation unit or into the cucurbit of the alembic. A Clevenger apparatus, for example, is used. Distilled, reverse osmosis or ultrapure water is added until the exudate is covered and brought to a boil, said boil is maintained so that hydrodistillation takes place for a period of between 2 and 24 hours.

The hydrodistillation flask or cucurbit is then drained, either hot or cold, to recover the residue mixed with the residual hydrodistillation water. The residue is then allowed to cool until it solidifies from 35° C., then the residual water is removed.

As an alternative to a hydrodistillation operation carried out at atmospheric pressure, a vacuum hydrodistillation or even vapodistillation operation known to the person skilled in the art can be carried out by bringing the exudate into contact with steam.

At the end of the hydrodistillation or vapodistillation operations, the cooled residue can then be coarsely crushed, optionally dried to eliminate the potential presence of residual water and to allow a powder to be obtained by grinding.

The solvent extraction of the residue powder thus obtained is carried out by bringing it into contact with a non-polar solvent, for example hexane, or a polar solvent, for example ethanol. Ethanol is preferred since it can be of natural origin, derived from agricultural resources such as for example beet or cereals. A mixture of solvents can also be used. The ratio between the residue powder and the solvent, or the solvent mixture, is at least 1 gram of residue powder per 4 milliliters of solvent, but can be increased up to 1 gram of residue powder per 100 milliliters of solvent for example. The mixture is stirred at a speed of between 50 and 700 rpm for at least 5 minutes at room temperature or at a temperature greater than 25° C. taking care, in the latter case, to use a reflux assembly to condense the solvent vapors in the reaction medium.

The solid residues are then recovered by centrifuging the medium for at least 20 minutes at between 2000 and 6000 rpm. Alternatively, the operation of recovering solid residues can be carried out by filtration.

The supernatant (centrifugation) or filtrate (filtration) is then evaporated to obtain a dry extract of residue in the form of a solid foam. Evaporation can be carried out under vacuum or at atmospheric pressure, by heating or at room temperature, or even cold.

Advantageously, the extraction operation can be repeated at least once, preferably twice, on the solid residues derived from the centrifugation or filtration operation previously indicated. The operation of evaporating the supernatant or the filtrate is then carried out once again. It is possible to combine the supernatants or filtrates obtained during the solid residue recovery steps and carry out another evaporation to recover as much unsolubilized solid residue as possible. This dry residue extract, itself obtained from hydrodistillation or vapodistillation residue of Canarium exudate is therefore advantageously an alcoholic or hydroalcoholic extract. A first form of the ingredient of the invention is then obtained.

Finally, if the ingredient is to be obtained in powder form, the dry extract derived from the extraction, solid residue separation and evaporation operations can be shaped by grinding.

The ingredient of the invention has healing and anti-inflammatory properties, as demonstrated in Examples 2, 3, 4 and 5. In the field of cosmetics, the healing properties are generally associated with skin repair and regeneration properties, as the skin regeneration process ensures the restructuring of the integrity and functions of affected tissues.

Also, the ingredient of the invention comprises at least one Canarium extract, said extract is a non-volatile fraction of Canarium exudate. Preferentially, the fraction comprises at least one triterpene, more preferentially a mixture of triterpenes. When the fraction constituting the extract and therefore the ingredient according to the invention comprises a mixture of triterpenes, this mixture mainly comprises neutral and/or acidic triterpenes, mainly lanosterol, β-amyrin and α-amyrin for neutral triterpenes, and elemolic acid, elemonic acid and tsugaric acid for acidic triterpenes.

Also, according to an object of the invention, the active ingredient according to the invention comprises or consists of a fraction comprising at least one neutral triterpene selected from lanosterol, β-amyrin, α-amyrin, and combinations thereof.

According to one object, the fraction comprises at least lanosterol and its content is at least equal to 55 milligrams per gram of ingredient or otherwise expressed, at least equal to 5.5% by weight.

When the ingredient comprises β-amyrin, said ingredient has a β-amyrin content at least equal to 85 milligrams per gram.

When the ingredient comprises α-amyrin, said ingredient has an α-amyrin content at least equal to 215 milligrams per gram.

As demonstrated in Example 3, neutral triterpenes play a role in the healing properties of the ingredient of the invention.

However, as demonstrated in Example 5, acidic triterpenes also play a role in the healing properties of the ingredient of the invention.

As a result, according to another object of the invention, the active ingredient according to the invention comprises or consists of a fraction comprising at least one acidic triterpene selected from elemolic acid, β-elemonic acid, tsugaric acid A, and combinations thereof.

When the ingredient comprises elemolic acid, said ingredient has an elemolic acid content at least equal to 20 milligrams per gram.

When the ingredient comprises β-elemonic acid, said ingredient has a β-elemonic content at least equal to 45 milligrams per gram.

When the ingredient comprises tsugaric acid A, said ingredient has a tsugaric acid A content at least equal to 25 milligrams per gram.

The triterpene mixture may also comprise at least one other minority triterpene selected from lupeol, uvaol, erythrodiol, ursolic acid, oleanolic acid and combinations thereof.

Thus, when the fraction comprises at least one neutral triterpene, this is advantageously selected from lanosterol, β-amyrin, α-amyrin, lupeol, uvaol, erythrodiol and combinations thereof. Very advantageously, the fraction comprises at least two, or at least three, or at least four, or at least five or at least six neutral triterpenes selected from lanosterol, β-amyrin, α-amyrin, lupeol, uvaol and erythrodiol.

When the fraction comprises at least one acidic triterpene, this is advantageously selected from elemolic acid, β-elemonic acid, tsugaric acid A, ursolic acid, oleanolic acid and combinations thereof. Very advantageously, the fraction comprises at least two, or at least three, or at least four, or at least five acidic triterpenes selected from elemolic acid, β-elemonic acid, tsugaric acid A, ursolic acid and oleanolic acid.

This shows that the non-volatile fraction of Canarium exudate, that is, an extract of Canarium exudate, in particular all the molecules constituting the extract derived from Canarium exudate, confers on the cosmetic ingredient its properties for combating inflammation, acne, excess sebum and blackheads, but also for improving skin healing and regeneration, as demonstrated in Examples 1 to 6.

Advantageously, the neutral and acidic triterpenes of the ingredient are not classified as irritant or allergenic according to EC regulation 1272/2008.

Advantageously, to give the ingredient of the invention optimum properties, the extract constituting it is the non-volatile fraction of exudate of the species Canarium schweinfurthii.

Preferentially, said fraction is an extract of the residue of hydrodistillation or vapodistillation of Canarium schweinfurthii exudate, more preferentially, said extract is an alcoholic or hydroalcoholic extract.

Finally, in a particularly advantageous manner, the active ingredient according to the invention is capable of being obtained by a method comprising the following steps:
hydrodistillation or vapodistillation of the exudate of Canarium schweinfurthii, and
obtaining a hydrodistillation or vapodistillation residue forming the non-volatile fraction of Canarium schweinfurthii, and
extracting the non-volatile fraction obtained with the solvent, said solvent being preferably a volatile solvent, and
extracting the non-volatile fraction obtained with the solvent, said solvent being a polar solvent other than water, or non-polar, preferentially the solvent extraction is carried out at least twice, more preferentially three times.

The method can further comprise an additional step of removing the solvent, if the solvent is volatile, for example by vacuum evaporation.

The active ingredient according to the invention can take several forms, in particular solid form, notably powder form.

When it is in solid form, the active ingredient according to the invention is preferentially made up exclusively of the fraction corresponding to the residue of hydrodistillation or vapodistillation of Canarium exudate, in particular of Canarium schweinfurthii. Preservatives or stabilizers may be added to facilitate the preservation and use of this active ingredient. Additionally, residual traces of water or extraction solvent may remain.

According to one variant, the active ingredient can dissolve in an oil phase or a non-polar solvent for use in liquid form.

The invention also relates to a cosmetic composition comprising the ingredient of the invention and in a form suitable for topical application (Example 7). In particular, the active ingredient according to the invention is preferentially used in compositions, comprising a cosmetically/dermatologically acceptable medium. The compositions are in different galenical forms, suitable for topical application to the skin.

These compositions can notably be in the form of oil-in-water emulsions, water-in-oil emulsions, multiple emulsions (water/oil/water or oil/water/oil) which may optionally be microemulsions or nanoemulsions, or in the form of solutions, suspensions, hydrodispersions, aqueous gels or powders. They can be more or less fluid and have the appearance of creams, balms, emulsions, gels, milks, cleansing oils or any other aspect of skin care cosmetic formulation.

Advantageously, the cosmetic composition comprises at least 0.1% by weight of the active ingredient according to the invention.

These compositions comprise, in addition to the active ingredient, a physiologically acceptable and preferably cosmetically and/or dermatologically acceptable medium, that is, one which does not cause feelings of discomfort that are unacceptable to the user such as redness, tightness or tingling.

The compositions according to the invention may contain as additive at least one compound selected from oils, waxes, surfactants, thickeners, gelling agents, colorants, preservatives, bulking agents, fragrances and mixtures thereof.

Examples of such additives are cited notably in the CTFA Dictionary (International Cosmetic Ingredient Dictionary and Handbook, published by the Personal Care Products Council).

Of course, the person skilled in the art will take care to choose any additional, active or non-active compounds, and their quantity, such that the advantageous properties of the mixture are not, or substantially not, altered by the envisaged additive.

These compositions are notably intended for use in improving skin quality, in particular to prevent and/or combat acne, prevent and/or combat blackheads, prevent and/or combat inflammation, prevent and/or combat excess sebum and improve skin healing.

Thus, the active ingredient and the compositions according to the invention, are particularly effective for topical skin treatment and in particular for an anti-inflammatory effect, including inflammation-related skin disorders, in particular psoriasis or eczema.

The active ingredient and the composition according to the invention are also of particular interest for improving skin healing, repair and regeneration, as demonstrated in the examples. Also, the active ingredient according to the invention has interesting healing properties.

As a result, the invention also has as its object an active ingredient according to the invention or a composition including it for use thereof for its healing effects on the skin, for its anti-inflammatory effects, in particular to combat inflammation of skin cells and/or sebocytes.

According to another object, the active ingredient and the composition according to the invention are also useful for preventing or treating acne, but also for combating excess sebum secretion. The invention is also aimed at a cosmetic use of the ingredient according to the invention to combat excess sebum secretion, including its consequences, notably but not limited to the appearance of blackheads. These spots, due notably to excess sebum and oxidation thereof, appear mainly on the face and the scalp, in an unsightly manner.

Thus, the object of the invention is also the cosmetic use of the active ingredient according to the invention or of the composition including it, by topical application, to combat blackheads, but also to treat them. The invention also aims to use the ingredient for its purifying, mattifying, soothing, detoxifying, protective, sebum-regulating, anti-blemish, anti-redness, anti-irritant and anti-itching effects.

The ingredient according to the invention is thus particularly intended for sensitive skin, notably atopic, irritated, weakened and reactive skin.

According to a last aspect, the method for extracting the extract constituting or contained in the active ingredient according to the invention can be obtained by any method comprising at least one step of hydrodistillation or vapodistillation of the exudate of Canarium, such as Canarium luzonicum, Canarium madagascariense, or Canarium schweinfurthii, in particular Canarium schweinfurthii, and recovering the non-volatile fraction, followed by extracting the non-volatile fraction obtained with the solvent, said solvent being a polar solvent other than water, or a non-polar solvent.

According to a particularly suitable embodiment, the active ingredient according to the invention is therefore obtained by implementing the following steps:
hydrodistillation or vapodistillation of the exudate of Canarium schweinfurthii, and
obtaining a hydrodistillation or vapodistillation residue forming the non-volatile fraction of Canarium schweinfurthii, and
extracting the non-volatile fraction obtained with the solvent, said solvent being preferably a volatile solvent, and
collecting solid residues by filtration or centrifugation, and
evaporating the filtrate or supernatant if the solvent used is volatile to produce an active ingredient powder.

The invention is now shown by non-limiting examples of extracts and of compositions according to the invention and by results.

### Example 1: Method for Preparing a Non-Volatile Fraction of Canarium schweinfurthii Exudate

### Hydrodistillation:

The exudate, or oleoresin, of Canarium schweinfurthii which has already been collected after non-destructive incision, is stored at −20° C. and split into pieces. The pieces of exudate are introduced into the cucurbit pot of the alembic. Demineralized water is added to the cucurbit until the exudate is covered. The water is then brought to the boil, said boil is maintained for a hydrodistillation time of 8 hours.

The residual water present in the cucurbit is then drained and the pot containing the hydrodistillation residue is discharged then cooled to room temperature for solidification of the residue. The solid residue is then recovered.

The residue is coarsely crushed, the residual moisture removed by drying at 40° C. for 12 hours and finally grinding is carried out until a residue powder is obtained.

### Solvent Extraction:

Solvent extraction of the residue powder thus obtained is carried out by bringing it into contact with 96° ethanol in a ratio of 1 gram of powder to 4 milliliters of ethanol. The reaction medium is stirred at a speed of 500 rpm for 45 minutes at room temperature.

The medium is then centrifuged for 20 minutes at 4400 rpm. The supernatant is then removed and evaporated under vacuum at a pressure of less than 60 mbar at 25° C. to obtain a dry extract.

The extraction, centrifugation and solvent removal operations are repeated twice.

Finally, the dry extract is ground to obtain the ingredient of the invention in powder form.

### Example 2: Composition and Anti-Inflammatory and Healing Properties of the Ingredient Derived from a Non-Volatile Fraction of Canarium schweinfurthii Exudate

The ingredient in powder form obtained in Example 1 was analyzed. The identification of its main compounds and their quantification were carried out by gas and liquid chromatography, combined with mass spectrometry and UV-visible detection. The main compounds of the injection and their content by weight are shown in Table 1 below.

TABLE 1
Triterpene composition of the ingredient

| | % by weight |
|---|---|
| Lanosterol (neutral triterpene) | 9.56 ± 1.91 |
| β-amyrin (neutral triterpene) | 10.67 ± 0.88 |
| α-amyrin (neutral triterpene) | 23.91 ± 0.89 |
| Elemolic acid (acidic triterpene) | 3.07 ± 0.38 |
| B-elemonic acid (acidic triterpene) | 5.47 ± 0.21 |
| Tsugaric acid A (acidic triterpene) | 3.58 ± 0.15 |

### Demonstrating the Healing Effect

Tests are carried out by measuring the ability of a population of monolayer cells grown in the absence and presence of the ingredient to close an artificial wound created by scratching (scratch technique). This technique manually creates an artificial cell-free space in a cell monolayer. The ability of the cells to fill this space is then assessed by monitoring the evolution of the wound surface.

Four solutions are tested. Each is obtained by dilution in a culture medium and obtaining a final concentration of 1% ethanol. The control solution does not contain the ingredient. The second, third and fourth solutions differ in the concentration of the ingredient (respectively 1.4 μg/mL, 3.5 μg/mL and 7.0 μg/mL).

On D0 (Day 0), an artificial wound is created by scratching the cell layer. After washing to remove cell debris, the samples receive the DMEM (Dulbecco's Modified Eagle Medium) test medium comprising one of the solutions tested (ethanol control, solution 1, solution 2 or solution 3). For each wound, a specific area is selected using an inverted microscope and recorded after capturing the image using a camera. After acquisition and recording of the images, the cultures are returned to the oven and incubated at 37° C. for 2 to 3 days. The media are renewed every day after twice-daily measurement of the wounds using an image analysis system.

The tests are carried out on HaCaT human epidermal keratinocytes. The solutions tested are introduced into contact with epidermal cells immediately after the formation of superficial wounds.

For each sample, the wound surface is measured by image analysis at D0, D1 and D2, giving results at T1 (8 h), T2 (24 h), T3 (32 h) and T4 (48 h) respectively. The surface area of the wounds at the different times T1, T2, T3 and T4 is evaluated as a percentage of the initial surface area of the wound considered (that is, 100% at TO). An associated healing percentage is also evaluated.

The results are presented in Table 2 below.

TABLE 2
Results of healing activity of the ingredient of the invention according to three different concentrations

| | T0 | | T1 (8 h) | | T2 (24 h) | | T3 (32 h) | | T4 (48 h) | |
|---|---|---|---|---|---|---|---|---|---|---|
| | Surf* | % He** | Surf* | % He** | Surf* | % He** | Surf* | % He** | Surf* | % He** |
| Ethanol | 100 ± 0 | 0 ± 0 | 84 ± 3 | 15.87 ± 2.71 | 51 ± 3 | 48.96 ± 1.29 | 35 ± 3 | 64.57 ± 1.03 | 0 ± 0 | 100.00 ± 5.53 |
| control | | | | | | | | | | |
| Sol 1 | 100 ± 0 | 0 ± 0 | 79 ± 4 | 20.49 ± 2.88 | 28 ± 5 | 71.88 ± 2.62 | 0 ± 0 | 99.93 ± 8.50 | 0 ± 0 | 100.00 ± 8.41 |
| (1.4 | | | | | | | | | | |
| μg/mL) | | | | | | | | | | |
| Sol 2 | 100 ± 0 | 0 ± 0 | 82 ± 1 | 17.53 ± 1.38 | 25 ± 4 | 74.52 ± 4.19 | 0 ± 0 | 99.98 ± 1.07 | 0 ± 0 | 100.00 ± 1.09 |
| (3.5 | | | | | | | | | | |
| μg/mL) | | | | | | | | | | |
| Sol 3 | 100 ± 0 | 0 ± 0 | 81 ± 3 | 18.68 ± 0.74 | 23 ± 9 | 76.45 ± 11.11 | 0 ± 0 | 100 ± 14.27 | 0 ± 0 | 100 ± 14.27 |
| (7.0 | | | | | | | | | | |
| μg/mL) | | | | | | | | | | |

*Surface area of wounds expressed as a % of the initial surface area of the wound considered
**Percentage of healing

There is a substantial increase in the healing kinetics of wounds to which the solutions containing the ingredient of the invention are applied, compared with the ethanol control. The healing effect is comparable for the three solutions 1, 2 and 3. The differences recorded at times T2 and T3 for the three solutions 1, 2 and 3 are statistically significant with respect to the ethanol control. Solutions 1, 2 and 3 ensure complete closure of the artificial wound at T3 (32 h).

These results show that the ingredient of the invention has a noticeable healing effect and is therefore capable of significantly increasing the kinetics of skin repair.

### Demonstrating the Anti-Inflammatory Effect

This test consists of evaluating the production of inflammatory mediators by human keratinocytes in response to irritant stress. The method is based on measuring the release rate of interleukin 6 (IL-6) by keratinocytes cultured in the presence of the ingredient and stimulated by UV light.

Five solutions are tested. Each is obtained by dilution in a culture medium and obtaining a final concentration of 1% ethanol. The control solution does not contain the ingredient. The second, third and fourth solutions differ in the concentration of the final residue (respectively 1.4 μg/mL, 3.5 μg/mL and 7.0 μg/mL). The fifth solution is dexamethasone (Dexa) (SIGMA Ref D1756) which is tested at 100 UM as a positive control.

On D0, 48 hours after seeding, the cells are rinsed with phosphate-buffered saline (PBS) and then exposed to UVB through the PBS. Immediately after irradiation, the PBS is replaced by the DMEM (Dulbecco's Modified Eagle Medium) test medium comprising the solution to be tested. The cells are returned to 37° C. and incubated for 24 hours.

On D1 (T 24 h), the incubation media are collected and the level of IL-6 is measured using the ELISA assay kit (IL-6 HIGH SENSITIVITY ELISA KIT, ENZO). At the same time, the corresponding cell layers are rinsed with Hank's Balanced Salt Solution (HBSS) and processed for cellular protein assay. A NaOH solution is added to each sample. After incubation, the cell extracts are collected and frozen at −20° C. to await assay with the BCA Protein Assay kit (PIERCE®).

The crude levels of IL-6 (pg per sample) are deduced by interpolation from the standard curve established with the IL-6 standard in the kit. The average of the IL-6 levels is calculated for each sample.

The protein level (μg per sample) is deduced by interpolation from the standard curve established with a bovine serum albumin standard in order to express the IL-6 level in relation to the average protein level. The average level is calculated for each sample.

The average IL-6 level is related to the average protein level. The final values are expressed in pg/mg·prot. and as percentages with respect to the percentage of IL-6 induced in the ethanol control. The results are presented in Table 3 below.

TABLE 3
Results of anti-inflammatory activity of the ingredient of the invention
according to three different application concentrations

| | Induced IL-6 | |
|---|---|---|
| | (pg/mg prot.) | Induced IL-6 (%) |
| Ethanol control | 1305.14 | 100% |
| Sol 1 (1.4 μg/mL) | 643.87 | 49% |
| Sol 1 (3.5 μg/mL) | 602.05 | 46% |
| Sol 1 (7.0 μg/mL) | 546.65 | 42% |
| Dexa (100 μM) | 105.10 | 8% |

The IL-6 levels recorded for solutions containing the ingredient of the invention are significantly less than the IL-6 levels recorded for the ethanol control. The differences observed are statistically significant. It can also be seen that the results are dependent on the concentration of the ingredient. Under the same experimental conditions, the Dexa sample inhibits UV-induced IL-6 release by 92% which confirms the good responsiveness of the system and validates these tests.

It has thus been demonstrated that the ingredient of the invention has a significant anti-inflammatory effect on, at least, IL-6 production.

### Example 3: Healing Properties of Neutral Triterpenes Extracted from the Ingredient Derived from a Non-Volatile Fraction of Canarium schweinfurthii Exudate

An extract rich in neutral triterpenes is prepared from the ingredient obtained in Example 1 by separating the ingredient on silica gel using open-tubular liquid chromatography, followed by evaporation of the fraction rich in neutral triterpenes. The triterpene composition of the extract thus obtained is shown in Table 4 below.

TABLE 4
Triterpene composition of the neutral triterpene-
rich extract of the ingredient.

| | % by weight |
|---|---|
| Lanosterol (neutral triterpene) | 15.89 ± 0.14 |
| β-amyrin (neutral triterpene) | 12.72 ± 0.39 |
| α-amyrin (neutral triterpene) | 28.59 ± 0.75 |
| Elemolic acid (acidic triterpene) | 0 |
| β-elemonic acid (acidic triterpene) | 0 |
| Tsugaric acid A (acidic triterpene) | 1.57 ± 0.01 |

### Demonstrating the Healing Effect

The tests are carried out in the same way as explained in Example 2.

The results are presented in Table 5 below.

TABLE 5
Results of healing activity of the neutral triterpene-rich extract of the ingredient
of the invention according to three different application concentrations

| | T0 | | T1 (8 h) | | T2 (24 h) | | T3 (32 h) | | T4 (48 h) | |
|---|---|---|---|---|---|---|---|---|---|---|
| | Surf* | % He** | Surf* | % He** | Surf* | % He** | Surf* | % He** | Surf* | % He** |
| Ethanol | 100 ± 0 | 0 ± 0 | 84 ± 3 | 15.87 ± 2.71 | 51 ± 4 | 48.96 ± 1.29 | 35 ± 3 | 64.57 ± 1.03 | 0 ± 0 | 100.00 ± 5.53 |
| control | | | | | | | | | | |
| Sol 1 | 100 ± 0 | 0 ± 0 | 90 ± 3 | 10.53 ± 2.70 | 48 ± 4 | 51.91 ± 3.37 | 26 ± 2 | 74.24 ± 0.37 | 0 ± 0 | 100.00 ± 2.37 |
| (1.0 | | | | | | | | | | |
| μg/mL) | | | | | | | | | | |
| Sol 2 | 100 ± 0 | 0 ± 0 | 89 ± 3 | 10.80 ± 3.02 | 43 ± 4 | 57.11 ± 4.64 | 22 ± 2 | 77.82 ± 2.33 | 0 ± 0 | 100.00 ± 1.07 |
| (2.5 | | | | | | | | | | |
| μg/mL) | | | | | | | | | | |
| Sol 3 | 100 ± 0 | 0 ± 0 | 85 ± 2 | 14.97 ± 1.67 | 34 ± 3 | 65.77 ± 4.60 | 14 ± 2 | 85.66 ± 5.41 | 0 ± 0 | 100 ± 5.35 |
| (5.0 | | | | | | | | | | |
| μg/mL) | | | | | | | | | | |

*Surface area of wounds expressed as a % of the initial surface area of the wound considered
**Percentage of healing

There is also an increase in the healing kinetics of wounds to which solutions containing the ingredient of the invention are applied, however, this effect is less marked than with the fraction according to Example 2. The healing effect depends on the concentration of the solutions used. The differences recorded at time T2, particularly for solutions 2 and 3, and time T3 for all three solutions are statistically significant with respect to the ethanol control. Solutions 1, 2 and 3 also ensure complete closure of the artificial wound at T4 (48 h).

These results show that the neutral triterpenes of the active agent of the invention participate in the healing effect of the ingredient, however, the efficacy is less marked with respect to the fraction according to Example 2 demonstrating that the neutral triterpenes are not solely responsible for the healing effect of the ingredient according to the invention.

### Example 4: Comparison of the Healing Properties of the Ingredient Derived from a Non-Volatile Fraction of Canarium schweinfurthii and the Ingredient Derived from a Non-Volatile Fraction of a Residue of Canarium luzonicum Exudate

The ingredient in powder form derived from the exudate of Canarium schweinfurthii is obtained according to Example 1 and the ingredient in powder form derived from the exudate of Canarium luzonicum is obtained according to the same operating conditions as those of Example 1 from an exudate of Canarium luzonicum.

The triterpene composition of the two ingredients is shown in Table 6 below, together with the variability results obtained from a plurality of ingredient samples.

TABLE 6
Triterpene composition of ingredients derived from
Canarium schweinfurthii and Canarium luzonicum

| | Canarium schweinfurthii | | Canarium luzonicum | |
|---|---|---|---|---|
| | (% by weight of | | (% by weight | |
| | residue from | Variability (% | of residue from | Variability (% |
| | Example 1) | by weight) | Example 1) | by weight) |
| Lanosterol (neutral | 9.56 ± 1.91 | between 6.62 | 2.85 ± 0.37 | between 2.12 |
| triterpene) | | and 10.66 | | and 3.57 |
| β-Amyrin (neutral | 10.67 ± 0.88 | between 7.83 | 19.49 ± 0.57 | between 18.37 |
| triterpene) | | and 14.57 | | and 20.61 |
| α-Amyrin (neutral | 23.91 ± 0.89 | between 18.99 | 28.32 ± 0.66 | between 27.03 |
| triterpene) | | and 29.15 | | and 29.61 |
| Elemolic acid | 3.07 ± 0.38 | between 1.45 | 9.37 ± 0.47 | between 8.45 |
| (acidic triterpene) | | and 3.85 | | and 10.29 |
| β-elemonic acid | 5.47 ± 0.21 | between 2.49 | 4.48 ± 0.22 | between 4.05 |
| (acidic triterpene) | | and 6.69 | | and 4.91 |
| Tsugaric acid A | 3.58 ± 0.15 | between 1.02 | 2.06 ± 0.11 | between 1.84 |
| (acidic triterpene) | | and 3.87 | | and 2.27 |

### Comparison of the Healing Effect

The tests are carried out on both ingredients in the same way as explained in Example 2.

The results are presented in Table 7 below, based on tests on three wounds and are expressed as a percentage of healing.

TABLE 7
Comparison of results of healing activity of ingredients derived respectively from exudates of Canarium schweinfurthii
and Canarium luzonicum according to two different application concentrations

| | T0 | | T1 (8 h) | | T2 (24 h) | | T3 (32 h) | | T4 (48 h) | |
|---|---|---|---|---|---|---|---|---|---|---|
| | C.S* | C.L* | C.S* | C.L* | C.S* | C.L* | C.S* | C.L* | C.S* | C.L* |
| Sol 1 | 0 ± 0 | 0 ± 0 | 25.01 ± 0.42 | 21.02 ± 0.23 | 100.00 ± 3.44 | 85.66 ± 8.14 | 100.00 ± 3.44 | 100.00 ± 6.23 | 100.00 ± 3.44 | 100.00 ± 6.23 |
| (0.014 | | | | | | | | | | |
| μg/mL) | | | | | | | | | | |
| Sol 2 | 0 ± 0 | 0 ± 0 | 24.77 ± 0.72 | 24.99 ± 0.56 | 100.00 ± 4.11 | 80.31 ± 4.11 | 100.00 ± 4.11 | 100.00 ± 2.35 | 100.00 ± 4.11 | 100.00 ± 2.35 |
| (0.07 | | | | | | | | | | |
| μg/mL) | | | | | | | | | | |

C.S*: Canarium schweinfurthii
C.L*: Canarium luzonicum

While solutions 1 and 2 derived from Canarium schweinfurthii and Canarium luzonicum ensure complete closure of the artificial wound at T3 (48 h) at most, significantly greater healing kinetics were observed for the residue of Canarium schweinfurthii exudate, for which, and for the two concentrations tested, healing was 100% at T2, whereas it was 86% and 80% respectively for the residue of Canarium luzonicum exudate at the same time. The botanical origin also plays a major role in the efficacy of the ingredient according to the invention, notably for its healing properties.

As a result, the ingredient derived from the non-volatile fraction of Canarium schweinfurthii exudate has greater healing properties than the ingredient derived from the non-volatile fraction of Canarium luzonicum exudate.

### Example 5: Healing Properties of Acidic Triterpenes Extracted from the Ingredient Derived from a Non-Volatile Fraction of Canarium schweinfurthii Exudate

An extract rich in acidic triterpenes is prepared from the ingredient obtained in Example 1 by separating the ingredient on silica gel using open-tubular liquid chromatography, followed by evaporation of the fraction rich in acidic triterpenes. The triterpene composition of the extract thus obtained is shown in Table 8 below.

TABLE 8
Triterpene composition of the acidic triterpene-
rich extract of the ingredient.

| | % by weight |
|---|---|
| Lanosterol (neutral triterpene) | 0 |
| β-amyrin (neutral triterpene) | 0 |
| α-amyrin (neutral triterpene) | 0 |
| Elemolic acid (acidic triterpene) | 9.28 |
| β-elemonic acid (acidic triterpene) | 21.64 |
| Tsugaric acid A (acidic triterpene) | 0 |

### Demonstrating the Healing Effect

The tests are carried out in the same way as explained in Example 2.

The results are presented in Table 9 below.

TABLE 8
Results of healing activity of the acidic triterpene-rich extract of the ingredient
of the invention according to three different application concentrations

| | T1 (8 h) | | T2 (24 h) | | T3 (32 h) | | T4 (48 h) | | T5 (56 h) | | T6 (72 h) | |
|---|---|---|---|---|---|---|---|---|---|---|---|---|
| | Surf* | % He** | Surf* | % He** | Surf* | % He** | Surf* | % He** | Surf* | % He** | Surf* | % He** |
| Ethanol | 95 ± | 5.00 ± | 84 ± | 15.60 ± | 83 ± | 17.43 ± | 58 ± | 41.67 ± | 41 ± | 58.89 ± | 14 ± | 86.19 ± |
| control | 1 | 0.98 | 6 | 5.82 | 7 | 6.98 | 12 | 12.53 | 17 | 17.55 | 20 | 20.045 |
| Sol 1 | 83 ± | 17.30 ± | 58 ± | 41.63 ± | 46 ± | 54.20 ± | 5 ± | 94.67 ± | 0 ± | 100.00 ± | 0 ± | 100.00 ± |
| (0.7 | 2 | 1.62 | 6 | 6.46 | 8 | 7.92 | 5 | 5.12 | 0 | 0 | 0 | 0 |
| μg/mL) | | | | | | | | | | | | |
| Sol 2 | 80 ± | 19.51 ± | 61 ± | 39.40 ± | 55 ± | 45.30 ± | 23 ± | 76.57 ± | 15 ± | 84.69 ± | 11 ± | 88.82 ± |
| (1.75 | 5 | 5.01 | 9 | 9.46 | 11 | 11.34 | 17 | 17.48 | 17 | 17.49 | 17 | 16.60 |
| μg/mL) | | | | | | | | | | | | |
| Sol 3 | 84 ± | 15.85 ± | 62 ± | 37.98 ± | 56 ± | 43.61 ± | 38 ± | 61.82 ± | 31 ± | 68.68 ± | 29 ± | 70.99 ± |
| (3.5 | 6 | 6.11 | 13 | 12.86 | 14 | 14.46 | 23 | 22.76 | 24 | 29.91 | 23 | 22.80 |
| μg/mL) | | | | | | | | | | | | |

*Surface area of wounds expressed as a % of the initial surface area of the wound considered
**Percentage of healing

It can be seen that the treatment of cells with the ingredient according to Example 5, in the range of concentrations tested (between 0.7 and 3.5 μg/mL), very significantly accelerates and stimulates wound healing.

These results also show that acidic triterpenes participate in the healing effect of the ingredient according to the invention, demonstrating once again that neutral triterpenes are not solely responsible for the healing effect of the ingredient according to the invention.

Also, these results confirm that the healing effect of the ingredient is linked to the intrinsic characteristics of the extract comprising the fraction derived from the residue of hydrodistillation or vapodistillation of Canarium exudate, in particular to the mixture of molecules making up the extract, comprising, but not limited to, acidic triterpenes and neutral triterpenes.

### Example 6: Anti-Sebum Effects of the Ingredient Comprising a Non-Volatile Fraction of Canarium schweinfurthii Exudate on Human Sebocytes

The aim of this test is to study the anti-sebum effect of the ingredient according to the invention using an in vitro cell model. An extract is prepared according to Example 2.

The procedure is as follows. PCi-SEB human sebocytes derived from induced pluripotent stem (iPS) cells are prepared. The amplification and the maturation of PCi-SEB sebocytes is achieved by incubation in SEB medium for amplification for 2 days then in supplemented SEB medium for maturation for 3 days. The sebocytes are incubated for 24 hours in the presence of the ingredient according to the invention. Lipogenesis is induced by the addition of linoleic acid, then the sebocytes are incubated for 48 hours in the presence of the ingredient according to the invention.

In order to verify efficacy, the following biomarkers are measured: namely, intracellular neutral lipids (Nile Red test) and cellular proteins (Pierce BCA test).

The experimental plan is as follows:
1 batch (Control): untreated cells incubated in SEB medium (n=3)
1 batch (EtOH control): untreated cells incubated in SEB medium+EtOH (0.125%)] (n=3)
3 batches (Test element-treated): cells incubated in medium (SEB+EtOH (0.125%)) and treated with 3 concentrations of test element (n=3)
1 batch [PC]: cells incubated in SEB medium and treated with TOFA (20 μM)

The ingredient according to the invention was tested at 3 concentrations: C1=5 μg/mL, C2=15 μg/mL and C3=30 μg/mL

The level of neutral lipids was measured in control sebocyte cultures then treated by staining the cells with Nile Red. The values (fluorescence intensity) were reported at the average cellular protein level.

The results are presented in Table 10 below.

TABLE 10
Results on the sebum-regulation of the ingredient of the invention

| | | | | | | | TOFA |
|---|---|---|---|---|---|---|---|
| | | | | | | | positive |
| | | | | | | | control |
| | | | | | | | (5- |
| | | | | | | | (tetradecyl- |
| | | Negative | Ethanol | | | | oxy)-2-furoic |
| | | control | control | Ingredient of the invention | | | acid) |
| Nile Red Test | | — | EtOH (0.1%) | 5 μg/mL | 15 μg/mL | 30 μg/mL | 20 μM |
| Mean | | 8172 | 7103 | 5738 | 2601 | 1606 | 1679 |
| Standard deviation | | 939 | 538 | 448 | 901 | 1498 | 756 |
| Protein test | | — | EtOH (0.1%) | 5 μg/mL | 15 μg/mL | 30 μg/mL | 20 μM |
| Mean | | 0.576 | 0.573 | 0.573 | 0.0550 | 0.565 | 0.551 |
| Standard deviation | | 0.029 | 0.006 | 0.013 | 0.005 | 0.019 | 0.004 |
| Protein | Mean | 251.52 | 250.05 | 250.05 | 239.30 | 246.40 | 239.99 |
| (μg/well) | Standard | 13.47 | 2.85 | 6.10 | 2.47 | 8.81 | 1.65 |
| | deviation | | | | | | |
| Neutral lipids - Nile | | — | EtOH (0.1%) | 5 μg/mL | 15 μg/mL | 30 μg/mL | 20 μM |
| Red | | | | | | | |
| Nile-Ref | Mean | 32.63 | 28.42 | 22.93 | 10.86 | 6.39 | 7.01 |
| (URF/mg | Standard | 4.86 | 2.38 | 1.22 | 3.75 | 5.78 | 3.21 |
| prot.) | deviation | | | | | | |

| Neutral lipids (% of | 115 | 100 | 81 | 38 | 22 | 25 |
|---|---|---|---|---|---|---|
| control_{EtOH}) | | | | | | |
| Lipogenic effect (%) | | — | −19 | −62 | −78 | −75 |
| | n.s. | Stat. (vs. | p < 0.05 | p < 0.01 | p < 0.01 | p < 0.01 |
| | | Control_{EtOH}) | | | | |

The [Nile-Red] values are expressed in arbitrary units (fluorescence intensity (urf) per mg of cellular protein).

The results show a clear reduction in intracellular neutral lipid levels in cells treated with the ingredient according to the invention. This inhibitory effect is dose-dependent over the range of concentrations tested and the differences recorded in batches C1 (−19%, p≤0.05), C2 (−62%, p≤0.01) and C3 (−78%, p≤0.01) are statistically significant (Student's t-test) compared with the EtOH Control.

This shows the very strong inhibitory potential of the ingredient according to the invention with regard to lipogenesis. Furthermore, at the highest dose tested, the anti-sebum effect of the ingredient according to the invention is equivalent to that of TOFA (20 UM), thus demonstrating the anti-sebum properties and the ability of the ingredient according to the invention to combat acne and blackheads.

### Example 7: Cosmetic Composition Comprising the Ingredient of the Invention

An example of a cosmetic composition formulation is shown in Table 11 below. The name of each component as referenced in the INCI (International Nomenclature of Cosmetic Ingredients) list is shown in the Table, along with the associated weight percentage.

TABLE 11
Cosmetic composition formulation of the invention

| | INCI name | % by weight |
|---|---|---|
| Grape seed oil | VITIS VINIFERA SEED OIL | 20.0% |
| Cetearyl alcohol | CETEARYL ALCOHOL | 2.0% |
| Water | AQUA | QSP 100% |
| Sugar ester | SUCROSE STEARATE | 2.0% |
| Xanthan gum | XANTHAN GUM | 0.3% |
| Benzoic acid | BENZOIC ACID | 0.5% |
| C. schweinfurthii* | | 0.1 to 2.0% |

*Ingredient of the invention prepared according to Example 1

1. A cosmetic active ingredient comprising at least one extract of Canarium schweinfurthii, characterized in that said extract is a non-volatile fraction of Canarium schweinfurthii exudate.

2. The active ingredient according to claim 1, characterized in that the fraction comprises at least one triterpene.

3. The active ingredient according to claim 2, characterized in that the fraction comprises at least one neutral triterpene and/or at least one acidic triterpene.

4. The active ingredient according to claim 2, characterized in that the fraction comprises at least one neutral triterpene selected from lanosterol, β-amyrin, α-amyrin, lupeol, uvaol, erythrodiol and combinations thereof.

5. The active ingredient according to claim 2, characterized in that the fraction comprises at least one acidic triterpene selected from elemolic acid, β-elemonic acid, tsugaric acid A, ursolic acid, oleanolic acid and combinations thereof.

6. The active ingredient according to claim 1, characterized in that the fraction is a hydrodistillation or vapodistillation residue of Canarium schweinfurthii exudate.

7. The active ingredient according to claim 6, characterized in that the fraction is an alcoholic or hydroalcoholic extract of the residue of hydrodistillation or vapodistillation of Canarium schweinfurthii exudate.

8. The active ingredient according to claim 1, characterized in that the ingredient is obtained by a method comprising the following steps:
hydrodistillation or vapodistillation in water of Canarium schweinfurthii exudate,
obtaining a hydrodistillation or vapodistillation residue forming the non-volatile fraction of Canarium schweinfurthii, and
extracting the non-volatile fraction obtained with the solvent, said solvent being a polar solvent other than water, or a non-polar solvent, and
recovering solid residues by filtration or centrifugation, and evaporating the filtrate or supernatant and recovering the active ingredient powder.

9. The ingredient according to claim 8, characterized in that the solvent extraction is carried out at least twice on the solid residues, preferably three times.

10. The active ingredient according to claim 1, for use thereof for its repairing effects on the skin.

11. The active ingredient according to claim 1, for use thereof for its anti-inflammatory effects.

12. The active ingredient according to claim 11, for use thereof in combating inflammation of skin cells and/or sebocytes.

13. The active ingredient according to claim 1, for use thereof on the skin to prevent and/or combat acne and/or combat excess sebum secretion.

14. A cosmetic composition comprising at least 0.1 wt. % of an active ingredient according to claim 1.

15. Use of an ingredient according to claim 1, by topical application, to combat blackheads.
